(19) United States
(12) Patent Application Publication
(10) Pub. No.: US 2010/0119610 A1
(43) Pub. Date: May 13, 2010

(54) PACKAGED PEGYLATED GOLD NANOPARTICLES

(75) Inventors: CHRISTIAN L. SCHOEN, LOVELAND, CO (US); Shelley Coldiron, Salt Lake City, UT (US)

Correspondence Address: MCKEE, VOORHEES & SEASE, P.L.C., 801 GRAND AVENUE, SUITE 3200, DES MOINES, IA, 50309-2721, US

(73) Assignee: CONCURRENT ANALYTICAL, INC., SALT LAKE CITY, UT (US)

(21) Appl. No.: 12/545,139

(22) Filed: Aug. 21, 2009

Related U.S. Application Data
(60) Provisional application No. 61/113,812, filed on Nov. 12, 2008.

(51) Int. Cl. A61K 9/16; A61K 39/395; A61K 38/16; A61K 31/7088; A61P 35/00

(52) U.S. Cl. 424/490; 424/179.1; 514/12; 514/44 R; 977/810

(57) ABSTRACT

Gold nanoparticles conjugated to polyethylene glycol and active binding molecules such as antibodies, proteins, lectins and DNA are suspended in a water vehicle at concentration from 10⁷ to 10¹⁵ and then placed in a sealed container such as a centrifuge tube, and then the centrifuge tube is sealed in a film package that is non-air permeable.

### CROSS-REFERENCE TO RELATED APPLICATIONS

This application claims priority under 35 U.S.C. §120 to provisional application Ser. No. 61/113,812 filed Nov. 12, 2008, herein incorporated by reference in its entirety.

### BACKGROUND OF THE INVENTION

Gold particles and particularly gold nanoparticles of spheroidal or rod shape are of significant interest for many uses such as surface enhanced Raman spectroscopy (SERS) and for thermal treatment of cancer tumors. Many other uses also exist, particularly for gold nanoparticles, and in particular hydrophilic polyethylene glycol coated gold particles, i.e., analytical techniques, bioassays, etc.

One particular problem that has existed for such particles is presentation of the packaged particle in a manner that provides polyethylene glycol nanoparticles (PGNP) that can be stored for long periods of time without adverse operability affects. As used herein adverse operability affects means that after storage the nanoparticles can be used for their intended purpose without sacrificing their utility.

In accordance with one of the objects of the invention as embodied and broadly described herein, the invention provides packaged stable products and compositions for shipping, storing and ready for use of shaped nanoparticles. The nanoparticles may be either spheres or rods in shape.

In another aspect the invention relates to packaged polyethylene glycol coated nanoparticles of gold (PGNP) of increased storage stability. That is to say they may be stored up to two weeks without sedimentation; they may be stored at room temperature as opposed to requiring refrigeration at 4° C.; and they may be stored without biological growth for up to two weeks.

In a further aspect the invention relates to a unique packaging system for PGNP particles whether shaped as spheres or rods.

Additional advantages of the invention will be set forth in part in the description which follows, and in part will be apparent from the description, or may be learned by practice of the invention. The advantages of the invention will be realized and obtained by means of the elements in combinations particularly pointed out in the appended claims. It is to be understood that both the foregoing general description and the following detailed description are exemplary and explanatory only and are not restrictive of the invention as claimed.

### BRIEF SUMMARY OF THE INVENTION

Gold nanoparticles conjugated to polyethylene glycol and active binding molecules such as antibodies, proteins, lectins and DNA are suspended in a water vehicle at a concentration from 10⁷ to 10¹⁵ of gold conjugated nanoparticle, and then placed in a non-air permeable sealed container such as a centrifuge tube and then the centrifuge tube itself is sealed in a film package that is non-permeable to air. The package can be stored at temperatures up to 70° C. for two weeks and will successfully resist biological growth. Cold temperature storage is thus avoided. The stability also exists over a wide range of pH, provides stability in up to two molar salt solutions, and provides particles that do not sediment out for long periods of time. Finally the particles are operable as neutral, negative or positively charged particles.

### BRIEF DESCRIPTION OF THE DRAWINGS

FIG. 1 is a perspective view of the fully packaged hydrophilic polyethylene glycol coated gold nanoparticles.

FIG. 2 is a view of the PGNP particle filled centrifuge tube which is placed inside the polyethylene package of FIG. 1.

FIG. 3 is a cross section through the filled sealed centrifuge tube shown in FIG. 2.

### DETAILED DESCRIPTION OF A PREFERRED EMBODIMENT

The applicant has developed and optimized packaged nanoparticles which may be either nanospheres or nanorods, specifically for use in biological, preclinical and in vivo applications. Preferably the particles are nanorods coated with a dense layer of hydrophilic polyethylene glycol (PEG) polymers that shield the gold surface of the nanoparticle and give the particles ultra long circulation times in vivo. In comparison with other commercially available PEG nanoparticles, those of the present invention have longer circulation times, greater packaged stability and overall superior product performance in vivo.

Typically gold nanoparticles, even those covered with hydrophilic polyethylene glycol need to be stored at colder temperatures, i.e., 4° C.; and often after standing they must be resuspended using sonication or vortexing before they can be used. These packaged particles also have good centrifugation properties and sedimentation properties. For purposes of comparison, Table 1 below shows comparative properties of prior art packaged GNPS and comparative properties of the PGNP gold particles of the present invention.

TABLE 1
Comparative Data

| | Adsorptive | |
|---|---|---|
| | gnps (prior art) | PGNPs of invention |
| pH | 5 to 9 | 3 to 11 |
| Temp | Always refrig to | Room temp storage up to 70° C. |
| | 4° C. | w/o biological growth for 2 weeks |
| Salt (saline) | Up to 100 mM | 1-2 M |
| Chemicals | No solvents | Most solvents |
| Centrifugation | 2 times | Up to 10 times |
| Charge control | None | Can be made −, +, or neutral |
| Sedimentation | 80 nm spheres | Did not sediment after two weeks |
| | sediment in 3 days | |

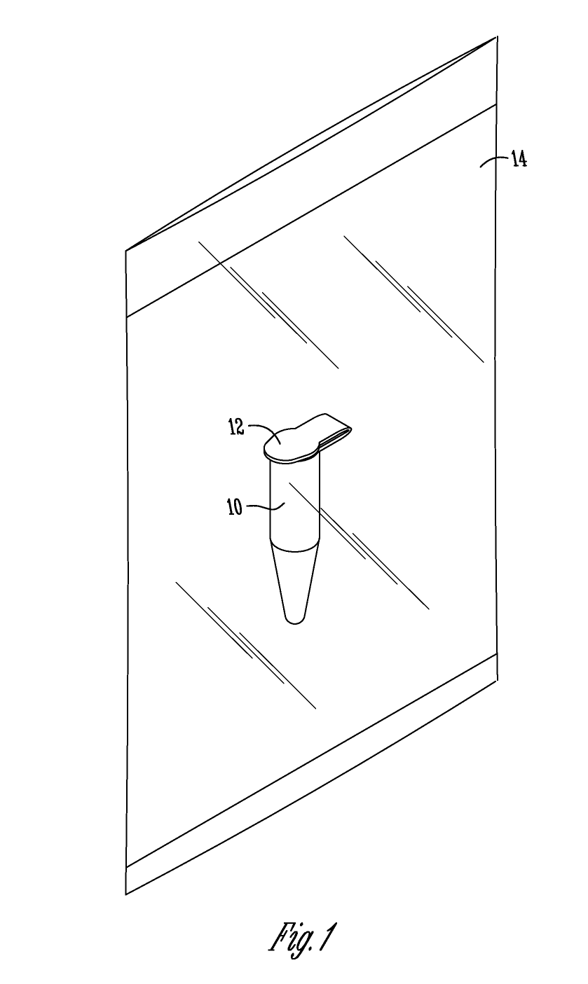

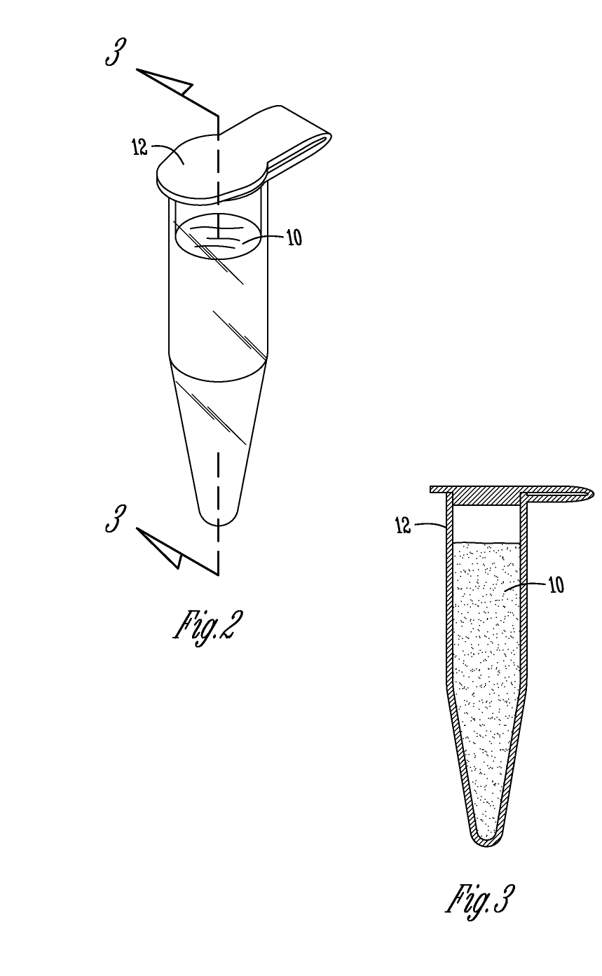

As best seen in FIG. 1, the gold particles 10 are collected inside of a centrifuge tube 12 and sealed, typically in phosphate buffer solution having less than 0.0001% ascorbic acid. The particles are gold nanoparticles which may vary in size from 10 nm nanoparticles to 100 nm nanoparticles. They usually are placed in a water vehicle at a concentration of from about 10⁷ to 10¹⁵ gold particles. Preferably to 10⁸ to 10¹² concentration. Gold particles may be spheres or rods, depending upon use. For particular advantages of nanorods see co-pending application, international publication WO/2006/065762-A2, the disclosure of which is incorporated herein by reference.

For the PGNP particles 10 packaged inside of the centrifuge tube 12 (which itself is sealed inside polyethylene package 14) specifications may be as follows:

TABLE 2
Specifications

| In batch size variation | <10% CV |
|---|---|
| Shape monodispersity | 95% nanorods |
| Surface charge (zeta) | +0 mV typ |
| Axial size | 10 nm |
| Wavelength coverage | 700-850 nm |
| pH | ~5 |

For use in in vivo heating of cancer tumors, 10 nm axial sized nanorods are used for wavelengths of 700 nm, 750 nm, 780 nm, 808 nm and 850 nm, since the 10 nm size offers the highest photo thermal conversion per gram. The packaged product can be available in highly concentrated solutions, for example 1 ml sizes, depending upon the size of the centrifuge tube utilized. Preferably the gold nanorods are shipped in PBS, with less than 0.0001% ascorbic acid, CTAB capping agent and unbound PEG.

In general, preferred PGNP particles are nanorods and may be made and packaged in accordance with the specifications shown in Table 3.

TABLE 3
Data Sheet

| Part # | Axial | | | | | | | | | | | | | | LSPR |
|---|---|---|---|---|---|---|---|---|---|---|---|---|---|---|---|
| Nanorod | Di- | | Peak | | Peak | | | | | | | LSPR | SSPR | Peak | Line- |
| (Axial | am- | Long | LSPR | OD | SSPR | OD | | Wt | | | Molar- | Molar | Molar | LSPR | width |
| DiamPeak | eter | Size | Wave | LSPR | Wave | SSPR | | conc. | | | ity | Ext. (M- | Ext. (M- | Accuracy | 80% |
| LSPR) | (nm) | (nm) | (nm) | (AU) | (nm) | (AU) | NPS/ml | (μgml) | Wt % | PPM | (pM) | (cm−1) | (cm−1) | (nm) | (nm) |
| 30-PM-850 | 10 | 45 | 850 | 50.0 | 512 | 12.50 | 2.6E+13 | 1786.7 | 0.1786% | 1786 | 43699 | 1.14E+09 | 2.86E+08 | +/−10 | 100 |
| 30-PM-808 | 10 | 41 | 808 | 50.0 | 512 | 12.50 | 2.9E+13 | 1805.6 | 0.1806% | 1806 | 48943 | 1.02E+08 | 2.55E+08 | +/−10 | 75 |
| 30-PM-780 | 10 | 38 | 780 | 50.0 | 512 | 12.50 | 3.1E+13 | 1745.8 | 0.1746% | 1746 | 50982 | 9.81E+05 | 2.45E+08 | +/−10 | 65 |
| 30-PM-750 | 10 | 35 | 750 | 50.0 | 512 | 12.50 | 3.2E+13 | 1670.7 | 0.1671% | 1671 | 53199 | 9.40E+08 | 2.35E+08 | +/−10 | 50 |
| 30-PM-700 | 10 | 29 | 700 | 50.0 | 512 | 12.50 | 3.3E+13 | 1483.6 | 0.1484% | 1484 | 55617 | 8.99E+08 | 3.25E+08 | +/−10 | 40 |

LSPR = Longitudinal SPR peak
SSPR = Axial SPR peak
Shape monodispersity (% rods) >95%
Size variation +/−10% (both dimensions)
Aspect ratio variation = Peak LSPR accuracy/96

The manner of treating the gold nanoparticles in order to conjugate them with polyethylene glycol is known, see Huff, Terry B., et al., “Controlling the Cellular Uptake of Gold Nanorods”, Langmuir, 23:1596-1599 (2007).

Importantly, the packaged gold particles, whether nanorods or nanospheres, when prepared as herein described, and when packaged as herein described, have unusually advantageous properties in the sense of being capable of successful use, including in vivo use without adverse operability affects. That is to say, they resist adverse operability affects at pH's of from 3 to 11; they may be stored at temperatures up to 70° C. for 2 weeks; they are stable in one or more salt solutions; they have no adverse operability affects to particle charges and can be neutral/negative or positive; and there is no noticeable sedimentation in centrifuge tubes stored for up to two weeks after packaging; in fact they can be centrifuges up to ten times without significant sedimentation. For nanorods, the preferred particle size is 10 nm to 25 nm diameter on the short axis and on the long axis 20 nm to 80 nm. The spherical particles may range from 5 nm to 100 nm in diameter.

The package 14, is preferably polyethylene film package, but other polyolefin film packages may also be used.

It will be apparent to those skilled in the art that various modifications and variations can be made in the description and the packaging herein without departing from the scope or spirit of the invention. Other embodiments of the invention will also be apparent and it is intended that specification and examples be considered as exemplary only with the true scope and spirit of the invention being defined by the following claims.

What is claimed is:

1. A packaged gold nanoparticle which can be stored long periods of time without adverse operability affects, comprising:
gold nanoparticles conjugated to PEG and an active binding molecule material selected from the group consisting of antibodies, proteins, lectins, and DNA;
a water vehicle for said gold conjugated nanoparticles;
said water vehicle having a particle concentration of from 10⁷ to 10¹⁵ of said gold conjugated nanoparticles;
a non-air permeable sealed container surrounding the gold nanoparticle/water vehicle mixture; and
a sealed film package around the non-air permeable sealed container.

2. The packaged gold nanoparticles of claim 1 wherein the particles are nanospheres.

3. The packaged gold particles of claim 1 wherein the particles are nanorods.

4. The package particles of claim 1 wherein the sealed container is a centrifuge tube.

5. The package gold nanoparticles of claim 3, which resist adverse operability effects and are stable at pH's of 3 to 11.

6. The package gold nanoparticles of claim 3, which are stable and resist biological growth and temperatures up to 70° C., stored for two weeks.

7. The package gold nanoparticles of claim 3 which are stable without adverse operability effects in 1 to 2 molar salt solutions.

8. The packaged gold nanoparticles of claim 1 which have a particle size of from 5 nm nanoparticles to 100 nm nanoparticles.

9. The packaged gold nanoparticles of claim 7 which are nanorods having an axial size of 10 n to 25 nm and a long axis size of 20 nm to 80 nm.

10. The packaged gold nanoparticles of claim 1 which are stable without adverse operability effects as either neutral, negative or positive charged particles.

11. The packaged gold nanoparticles of claim 1 which are stable without sedimentation for at least two weeks after packaging.

12. The packaged gold nanoparticles of claim 1 which can be centrifugal up to ten times without adverse operability effects.

13. The packaged gold nanoparticles of claim 1 which can be concentrated to optical densities between 50 and 5000.
